(19) United States
(12) Patent Application Publication
(10) Pub. No.: US 2018/0353126 A1
(43) Pub. Date: Dec. 13, 2018

(54) SYSTEM AND METHOD OF DIAGNOSING PEDIATRIC OBSTRUCTIVE SLEEP APNEA

(71) Applicant: Serenium, Inc., Palo Alto, CA (US)

(72) Inventors: David Gozal, Chicago, IL (US); Leila Gozal, Chicago, IL (US)

(73) Assignee: Serenium, Inc., Palo Alto, CA (US)

(21) Appl. No.: 15/779,018

(22) PCT Filed: Aug. 22, 2016
(86) PCT No.: PCT/US16/48009

§ 371 Date: May 24, 2018

Related U.S. Application Data
(60) Provisional application No. 62/207,780, filed on Aug. 20, 2015.

(51) Int. Cl. A61B 5/00; A61B 5/1455

(52) U.S. Cl. CPC A61B 5/4818; A61B 5/7267; A61B 5/14551

(57) ABSTRACT

One aspect of the present invention is to assess the performance of automated analysis of blood oxygen saturation (SpO2) recordings as a screening tool for OSAHS. As an initial step, statistical, spectral and nonlinear features are estimated to compose an initial feature set. Then, a fast correlation-based filter (FCBF) is next applied to search for the optimum subset. Finally, the discrimination power (OSAHS negative vs. OSAHS positive) of three pattern recognition algorithms is assessed: linear discriminant analysis (LDA), quadratic discriminant analysis (QDA) and logistic regression (LR). According to another aspect of the invention, oximetry is used to determine the OSAHS severity in children. For testing the severity of OSAHS, first spectral analysis is conducted to define and characterize a frequency band of interest in SpO2. Then the spectral data is combined with 3% oxygen desaturation index (ODI3) by means of a multi-layer perceptron (MLP) neural network, in order to classify children into one of the three OSAHS severity groups.

### PRIORITY

This application claims priority from U.S. Provisional Patent Application No. 62/207,780, filed Aug. 20, 2015, the disclosures of which are incorporated herein.

### FIELD OF THE INVENTION

This invention relates to methods, systems, and apparatus for sleep apnea monitoring. Specifically, it relates to methods, systems, and apparatus using oximetry for screening pediatric obstructive sleep apnea-hypopnea and predicting its severity.

### BACKGROUND OF THE INVENTION

Obstructive sleep apnea-hypopnea syndrome (OSAHS) is characterized by repetitive occlusion of the upper airway during sleep, causing intermittent cessations of breathing (apneas) or reduction in airflow (hypopneas). Events of apnea are accompanied by hypoxemia and bradycardia. They are often terminated in arousals, and the resulting sleep fragmentation can lead to excessive daytime sleepiness. As a result, OSAHS has been pointed out as a major public health concern. Additionally, long-term effects are related to the cardiovascular system, including hypertension, arrhythmias, congestive heart failure and cerebrovascular disease. Childhood OSAHS is also a highly prevalent but under-diagnosed condition. According to the American Academy of Pediatrics, OSAHS affects 1% to 5% of children in the general pediatric population. Untreated OSAHS has been associated with negative consequences in the development and performance of infants and young children, reducing overall health and quality of life, while increasing healthcare use and associated costs.

Pediatric OSAHS has also emerged as a frequent and concerning medical condition in the past 2-3 decades. It too is characterized by an abnormal breathing pattern during sleep that includes the recurrence of apneas (complete airflow cessation) and hypopneas (airflow limitation), caused by total or partial upper airway obstruction, respectively. Inadequate gas exchange characterized by repetitive hypoxia, hypercapnia, and accompanied by arousal episodes during the night has been suggested as the cause for serious comorbidities related to central nervous system and cardiovascular and metabolic system. Consequently, several daytime symptoms related to OSAHS, such as cognitive and behavioral irregularities as well as atypical growth are frequently present and reported by parents. Furthermore, the prevalence of OSAHS in children is high, with studies reporting up to 5.7% among general pediatric population.

The “gold standard” approach to diagnose OSAHS in children is overnight polysomnography (PSG). However, PSG has several limitations since it is both complex and costly due to the high number of physiological signals that need to be recorded. It must be performed in a special sleep unit and under supervision of a trained technician. PSG monitors different physiological recordings such as electrocardiogram (ECG), electroencephalogram (EEG), electrooculogram (EOG), electromyogram (EMG), oxygen saturation, abdominal ventilatory effort and snoring. These recordings must be subsequently analyzed by a medical expert to obtain a final diagnosis. Despite its high diagnostic performance, PSG presents some drawbacks since it is complex, expensive and time-consuming. Additionally, all the PSG signals need offline inspection in order to derive the apnea-hypopnea index (AHI), which is used to establish whether OSAHS is present and its severity. Moreover, children often do not tolerate well the equipment involved in PSG. As a result, research recently has focused on the development of alternative and simpler diagnostic techniques, such us the use of medical systems based on nocturnal pulse oximetry. An interesting approach is the analysis of single-channel sleep-related recordings, which reduces cost and complexity. In this regard, automated processing of oximetry signals is a promising alternative due to its reliability, simplicity, and suitability for children.

Nocturnal pulse oximetry allows to monitor respiratory dynamics during sleep by measuring blood oxygen saturation (SpO2). This recording provides useful information about OSAHS. Events of apnea are characterized by a decrease in the SpO2 value, which reflects airflow reduction and hypoxemia. Subsequently, respiration is restored and the saturation value increases until its baseline level. As a result, SpO2 signals from OSAHS patients tend to be more unstable than those from control subjects due to the recurrence of apneas during sleep. This different behavior can be exploited to diagnose OSAHS. Diverse methodologies have been proposed to perform OSAS diagnosis from SpO2 data. The simplest one is visual inspection. However, it is tedious and subjective. Therefore, automated analysis of SpO2 data would be desirable. Conventional oximetry indices represent a first approach for this purpose. These indices are the oxygen desaturation index over 2% (ODI2), 3% (ODI3) and 4% (ODI4), and the cumulative time spent below 90% of saturation (CT90). However, improved OSAHS diagnosis from SpO2 recordings is possible by using more advanced computer-implemented signal processing methods.

Additionally, one common approach has been the study of the diagnostic ability of reduced sets of signals derived from those involved in PSG, such as electrocardiography (ECG), photoplethysmography (PPG), airflow (AF), or SpO2. Particularly, frequency and time domain analyses of ECG-derived signals showed utility in pediatric OSAHS diagnosis. Moreover, the analysis of pulse transit time variability from PPG was successfully used to classify time segments into apneic or non-apneic, as well as children into normal subjects and OSAHS patients. Additionally, a recent study reported high diagnostic ability when combining the oxygen desaturation index (ODI) from SpO2 with spectral information from AF. Finally, spectral, nonlinear, and statistical features from SpO2 and pulse rate variability (PRV) recordings were obtained and successfully combined to establish OSAHS in children.

Related art includes U.S. patent application Ser. No. 10/947,983 which discloses a method for diagnosing OSAS based on a tool for the predicting Apnea Hypopnea Index (AHI) using non-parametric analysis and bootstrap aggregation; U.S. patent application Ser. No. 11/122,278 which discloses a method for monitoring respiration involving processing plethysmography signals; and U.S. patent application Ser. No. 10/302,008 which discloses a computer-implemented method for patient monitoring based on processing signals to detect breathing patterns. Additionally, U.S. patent application Ser. No. 13/561,011 discloses a system and method for monitoring the severity of sleep apnea using oximetry and AHI with a multilinear regression model or a multilayer perceptron network; and U.S. Pat. No. 8,862,195 pertaining to the detection of obstructive sleep apnea from oxygen saturation.

### SUMMARY OF THE INVENTION

As described above, childhood OSAHS is a highly prevalent condition that negatively affects health, performance and quality of life of infants and young children. Early detection and treatment improves neuropsychological and cognitive deficits linked with the disease. One aspect of the present invention is to assess the performance of automated analysis of blood oxygen saturation (SpO2) recordings as a screening tool for OSAHS. As an initial step, statistical, spectral and nonlinear features are estimated to compose an initial feature set. Then, a fast correlation-based filter (FCBF) is next applied to search for the optimum subset. Finally, the discrimination power (OSAHS negative vs. OSAHS positive) of three pattern recognition algorithms is assessed: linear discriminant analysis (LDA), quadratic discriminant analysis (QDA) and logistic regression (LR). Three clinical cutoff points commonly used in the practice for positive diagnosis of the disease were applied: apnea-hypopnea index (AHI) of 1, 3 and 5 events per hour (e/h). Testing of the methodology of the present invention reached 88.6% accuracy (71.4% sensitivity and 100.0% specificity, 100.0% positive predictive value, and 84.0% negative predictive value) in an independent test set using QDA for a clinical cut-off point of 5 e/h. These results suggest that SpO2 nocturnal recordings may be used to develop a reliable and efficient screening tool for childhood OSAHS.

According to another aspect of the invention, oximetry is used to determine the OSAHS severity in children. For testing this aspect of the invention, single-channel SpO2 recordings from 176 children were divided into three severity groups according to the apnea-hypopnea index (AHI): AHI<1 events per hour (e/h), 1≤AliI<5 e/h, and AliI≥5 e/h. For testing the severity of OSAHS, first spectral analysis is conducted to define and characterize a frequency band of interest in SpO2. Then the spectral data is combined with 3% oxygen desaturation index (ODI3) by means of a multi-layer perceptron (MLP) neural network, in order to classify children into one of the three OSAHS severity groups. Following this MLP multiclass approach, a diagnostic protocol with capability to reduce the need of polysomnography tests by 46% or more could be derived. Moreover, this aspect of the invention may be evaluated, in a binary classification task for two common AHI diagnostic cutoffs (AHI=1 e/h and AHI=5 e/h). Results showed that high diagnostic ability was reached in both cases (84.7% and 85.8% accuracy, respectively) outperforming the clinical variable ODI3 as well as other measures reported in recent studies. These results suggest that the information contained in SpO2 could be helpful in pediatric OSAHS severity detection.

### DETAILED DESCRIPTION OF THE EMBODIMENTS

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

While preferred embodiments of the present invention are shown and described, it is envisioned that those skilled in the art may devise various modifications of the present invention without departing from the spirit and scope of the appended claims.

### A. Automated Analysis of Nocturnal Oximetry as Screening Tool for Childhood Obstructive Sleep Apnea-Hypopnea Syndrome.

Previous oximetry-based studies in the context of OSAHS diagnosis assessed conventional indices, common statistics and conventional spectral features. Similarly, studies also used the information contained in pulse rate recordings from pulse oximetry. In the present invention, blood oxygen saturation (SpO2) recordings were analyzed. Statistical (first-to-fourth moments), spectral (amplitude, relative power and power distribution measures), nonlinear (irregularity, variability, and complexity measures), and conventional indices (number of desaturations from baseline) were computed. These metrics have been previously assessed in the context of OSAHS diagnosis both in adults and children. Fast correlation-based filter (FCBF) is proposed for feature selection. FCBF is a variable ranking methodology for feature selection independent of the classifier subsequently used in the classification stage. Linear discriminant analysis (LDA), quadratic discriminant analysis (QDA), and logistic regression (LR) are proposed for classification. QDA and LR are suitable alternatives to conventional LDA in binary classification problems but their performances have been weakly assessed in the context of childhood OSAHS. The methodology of the present invention detects complementary variables and provides general classification models useful as screening tools for OSAHS in children. One aspect of the invention was to design and assess several binary classifiers using different clinical cut-offs for OSAHS in order to analyze the screening ability at different severity thresholds. To achieve this goal, independent training and test datasets were analyzed to optimize the methodology.

### A.1 Subjects and Signals Under Study

A total of 176 children (97 boys and 79 girls) composed our dataset. All children were referred to the Pediatric Sleep Unit at the University of Chicago Medicine Corner Children's Hospital (Chicago, Ill., USA) due to clinical suspicion of suffering from OSAHS. Informed consents to participate in the study were obtained and the Institution's Ethical Review Committee approved the protocol.

Children's sleep was monitored using a digital polysomnography system (Polysmith; Nihon Kohden America Inc., CA, USA). SpO2 recordings from PSG (sampling frequency 25 Hz) were exported and processed offline. Artifacts were automatically removed by means of a preprocessing stage. SpO2 values equal to zero and differences between consecutive SpO2 samples 24% were considered artifacts.

The American Academy of Sleep Medicine rules were used to quantify sleep and cardiorespiratory events and derive the apnea hypopnea index (AHI), which averages the number of events per hour of sleep. Apnea was defined as the absence of oronasal airflow during at least 2 respiratory cycles. Hypopnea was defined as a decrease 250% lasting at least 2 respiratory cycles, leading to a desaturation 23% and/or an arousal. In the present study, the AHI-based clinical threshold was varied in order to assess the performance of the proposed methodology as a screening tool for OSAHS using commonly used cut-off points. AHI 2 1, 3, and 5 events per hour (e/h) from PSG were considered as OSAHS-positive. Table I displays demographic and clinical features of the dataset taking into account the proposed AHI cut-off thresholds for the disease. For each cut-off point, the whole population was randomly divided into independent training (60%) and test (40%) sets.

TABLE I
CLINICAL CHARACTERISTICS OF THE POPULATION USING
DIFFERENT CUT-OFF POINTS FOR OSAHS DIAGNOSIS

| | | |
|---|---|---|
| All children | OSAHS negative | OSAHS positive |

AHI 2 1 e/h

| | | | |
|---|---|---|---|
| N (n) | 176 | 30 | 146 |
| Age (years) | 6.95 ± 3.55 | 8.20 ± 3.28 | 6.70 ± 3.56 |
| Males (n) | 97 (55.11%) | 17 (56.67%) | 80 (54.79%) |
| BMI (kg/m²) | 20.62 ± 7.32 | 20.48 ± 6.77 | 20.64 ± 7.45 |
| AHI (e/h) | | 0.51 ± 0.31 | 10.70 ± 18.13 |

AHI 2 3 e/h

| | | | |
|---|---|---|---|
| N (n) | 176 | 79 | 97 |
| Age (years) | 6.95 ± 3.55 | 7.70 ± 3.23 | 6.36 ± 3.70 |
| Males (n) | 97 (55.11%) | 46 (58.23%) | 51 (52.58%) |
| BMI (kg/m²) | 20.62 ± 7.32 | 20.31 ± 6.73 | 20.87 ± 7.79 |
| AHI (e/h) | | 1.34 ± 0.80 | 15.17 ± 20.89 |

AHI 2 5 e/h

| | | | |
|---|---|---|---|
| N (n) | 176 | 105 | 71 |
| Age (years) | 6.95 ± 3.55 | 7.53 ± 3.44 | 6.10 ± 3.57 |
| Males (n) | 97 (55.11%) | 58 (55.23%) | 39 (54.93%) |
| BMI (kg/m²) | 20.62 ± 7.32 | 20.54 ± 6.70 | 20.74 ± 8.19 |
| AHI (e/h) | | 1.97 ± 1.33 | 19.31 ± 23.10 |

### A.2 Methodology

Firstly, each SpO2 recording was parameterized computing 17 features: time domain statistics (4), frequency domain statistics (6), conventional spectral features (3), nonlinear measures (3), and conventional oximetric indices (1). Then, a feature selection stage was applied using FCFB. An optimum feature subset was derived for each OSAHS cutoff. Finally, LDA, QDA, and LR models were composed for each feature subset. The training set was used for feature selection and model optimization whereas the test set was used for assessing all classifiers in an independent dataset.

A.2.1 Feature Extraction Stage

The following feature subsets were computed:

Time domain statistics. Mean (M1t), variance (M2t), skewness (M3t), and kurtosis (M4t) were derived from the data histogram of SpO2 amplitudes.

Frequency domain statistics. The distribution of power spectral density (PSD) amplitudes was parameterized by means of first-to-fourth statistical moments (M1f-M4f). In addition, the median frequency (MF) and spectral entropy (SE) were computed to quantify the degree of flatness of the power distribution.

Conventional spectral features. Total signal power (PT) and the peak amplitude (PA) and relative power (PR) in the apnea frequency band (0.021-0.040 Hz) were computed from the PSD.

Nonlinear measures. Sample entropy (SampEn, m=1, r=0.25), central tendency measure (CTM, r=1) and Lempel-Ziv complexity (LZC) were computed to quantify irregularity, variability and complexity.

Conventional oximetric indices [17]. Number of desaturations greater than or equal to 3% from baseline per hour of recording (ODI3).

SpO2 recordings were segmented into 1-min length epochs before computing the time domain features (each feature average value was subsequently obtained) whereas the PSD function was estimated using the Welch's method (15000-sample Hanning window, 50% overlap and 2¹⁴-points DFT).

A.2.2 Feature Selection Stage

FCBF computes the symmetric uncertainty (SU) to select relevant and non-redundant variables. SUi between the i-th input feature (Xi) and the AHI (Y) is defined as follows:

$${SU}_{i} ( {X}_{i} , Y ) = 2 \frac{{IG}_{i} ( {X}_{i} , Y )}{{H}_{i} ( {X}_{i} ) + H ( Y )} , i = 1 , … , p ,$$

where IG is the information gain and H is the well-known Shannon's entropy. In the first step, FCBF ranks features according to their relevance (the higher SUi the more relevant feature). Then, a threshold is used to discard irrelevant features. In this study, the log criterion was applied, where the cut-off is the SU value of the [N/log(N)]-th ranked feature. In the second step, redundant features are removed. In order to perform the redundancy analysis, SUij(featurei,featurej) between each pair of remaining ranked features (so that SUi 2 SUj) is computed. Then, feature j is removed if SUi,j≥SUi due to redundancy.

### A.2.3 Feature Classification Stage

Conventional statistical pattern recognition techniques were used for binary classification:

Linear discriminant analysis (LDA). Statistical classification algorithms based on discriminant analysis assume normality to model each class-conditional density function p(x|cⱼ) for input pattern x and class cj. If homoscedasticity is also presumed, i.e. all the class covariance matrices are equal (Σj=Σ), then the classification rule is called LDA and a linear decision threshold is assumed. Equation (2) shows the classification rule,

yⱼ(x)=μⱼ^{r}Σ⁻¹x−1/2μⱼ^{r}Σ⁻¹μⱼ+ln(P(cⱼ)),

where μj and Σ are the class cj mean vector and covariance matrix, respectively.

Quadratic discriminant analysis (QDA). In a more general context where it is not possible to presume homoscedasticity, the Bayes classification rule that minimizes the classification error function establishes a quadratic decision boundary between classes in the feature space. Equation (3) shows the classification rule under these assumptions,

yⱼ(x)=−1/2(x−μⱼ)^{T}Σⱼ⁻¹(x−μⱼ)−1/2ln(P(cⱼ))

Logistic regression (LR). No a priori normality and homoscedasticity of data are presumed. A binary LR classifier models the probability density function as a Bernoulli distribution. The maximum likelihood criterion is used to optimize the coefficients of the logistic model. Equation (4) shows the logistic classification function:

where fl is the vector of coefficients of the LR model.

$$y ( x , β ) = \frac{1}{1 + {e}^{- ( {β}_{0} + {β}_{1} {x}_{1} + … + {β}_{n} {x}_{n} )}} ,$$

### A.2.4 Statistical Analysis

The true positive rate (sensitivity, Se), true negative rate (specificity, Sp), proportion of positive tests that are true positive patients (positive predictive value, PPV), proportion of negative tests that are true negative subjects (negative predictive value, NPV), and percentage of children correctly classified (accuracy, Acc) were computed in order to assess the performance of each independent variable and optimum LDA, QDA, and LR models. Default classification thresholds of 0 (LDA and QDA) and 0.5 (LR) were applied.

### A.3 Results

### A.3.1 Training set

The proposed features were computed in order to compose the initial feature space. ROC analyses were carried out for each single feature to obtain their optimum classification thresholds in the training set. Next, optimum feature subsets were derived using FCFB. Table II shows the selected features for each diagnostic threshold. Model training of LDA, QDA, and LR classifiers was carried out.

### A.3.2 Test set

Table III summarizes the diagnostic performance of each single feature in the test set using the threshold derived from the training dataset. ODI3 achieved the highest performance in terms of accuracy (77.1%) using a threshold for OSAHS equal to 1 e/h, whereas PA reached the maximum accuracy (77.1%) applying a cut-off equal to 3 e/h and M2t, M1f, and PT reached the highest accuracy (82.9%) using a threshold equal to 5 e/h. Optimum pattern recognition models for each OSAHS cut-off were also assessed in the independent test set. Table IV summarizes the performance analysis. Using an AHI=1 e/h for positive OSAHS, the LR model composed of features from FCBF achieved an accuracy of 77.1% (91.4% Se, 8.3% Sp), whereas 72.9% (61.5% Se, 87.1% Sp) was reached using a cut-off equal to 3 e/h. The highest performance in terms of accuracy was achieved using a cutoff for OSAHS equal to 5 e/h, where QDA reached 88.6% accuracy (71.4% Se, 100.0% Sp).

TABLE II
OPTIMUM FEATURE SUBSETS USING FCBF FEATURE
SELECTION FOR EACH CUT-OFF POINT FOR OSAHS

| Optimum features | | cut-off | cut-off |
|---|---|---|---|
| (FCBF) | cut-off AHI = 1 e/h | AHI = 3 e/h | AHI = 5 e/h |
| Log criterion | M2t, M1f, Pr, PA, | M2t, M1f, PA, | M2t, Pr, PA, |
| | SampEn, ODI3 | SampEn, ODI3 | SampEn, ODI3 |

TABLE III
DIAGNOSTIC ASSESMENT OF EACH SINGLE VARIABLE FROM
THE INITIAL FEATURE SPACE IN THE TEST SET

| Performance | cut-off AHI = 1 e/h | | | | | cut-off AHI = 3 e/h | | | | | cut-off AHI = 5 e/h | | | | |
|---|---|---|---|---|---|---|---|---|---|---|---|---|---|---|---|
| (%) | Se | Sp | PPV | NPV | Acc | Se | Sp | PPV | NPV | Acc | Se | Sp | PPV | NPV | Acc |
| M1t | 41.4 | 100.0 | 100.0 | 26.1 | 51.4 | 51.3 | 77.4 | 74.1 | 55.8 | 62.7 | 71.4 | 71.4 | 62.5 | 79.0 | 71.4 |
| M2t | 60.3 | 83.3 | 94.6 | 30.0 | 64.3 | 71.8 | 80.7 | 82.4 | 69.4 | 75.7 | 89.3 | 78.6 | 73.5 | 91.7 | 82.9 |
| M3t | 53.5 | 58.3 | 86.1 | 20.6 | 54.3 | 87.2 | 41.9 | 65.4 | 72.2 | 67.1 | 60.7 | 52.4 | 46.0 | 66.7 | 55.7 |
| M4t | 55.2 | 50.0 | 84.2 | 18.8 | 54.3 | 59.0 | 45.2 | 57.5 | 46.7 | 52.9 | 50.0 | 50.0 | 40.0 | 60.0 | 50.0 |
| M1f | 48.3 | 91.7 | 96.6 | 26.8 | 55.7 | 66.7 | 80.7 | 81.3 | 65.8 | 72.9 | 89.3 | 78.6 | 73.5 | 91.7 | 82.9 |
| M2f | 50.0 | 83.3 | 93.6 | 25.6 | 55.7 | 69.2 | 77.4 | 79.4 | 66.7 | 72.9 | 89.3 | 71.4 | 67.6 | 90.9 | 78.6 |
| M3f | 34.5 | 50.0 | 76.9 | 13.6 | 37.1 | 69.2 | 41.9 | 60.0 | 52.0 | 57.1 | 50.0 | 35.7 | 34.2 | 51.7 | 41.4 |
| M4f | 34.5 | 50.0 | 76.9 | 13.6 | 37.1 | 69.2 | 41.9 | 60.0 | 52.0 | 57.1 | 50.0 | 50.0 | 40.0 | 60.0 | 50.0 |
| MF | 60.3 | 41.7 | 83.3 | 17.9 | 57.1 | 71.8 | 41.9 | 60.9 | 54.2 | 58.6 | 71.4 | 40.5 | 44.4 | 68.0 | 52.9 |
| SE | 53.5 | 58.3 | 86.1 | 20.6 | 54.3 | 46.2 | 71.0 | 66.7 | 51.2 | 57.1 | 64.3 | 57.1 | 50.0 | 70.6 | 60.0 |
| P_{T} | 48.3 | 91.7 | 96.6 | 26.8 | 55.7 | 66.7 | 80.7 | 81.3 | 65.8 | 72.9 | 89.3 | 78.6 | 73.5 | 91.7 | 82.9 |
| PA | 51.7 | 91.7 | 96.8 | 28.2 | 58.6 | 71.8 | 83.9 | 84.9 | 70.3 | 77.1 | 89.3 | 69.1 | 65.8 | 90.6 | 77.1 |
| P_{R} | 58.6 | 66.7 | 89.5 | 25.0 | 60.0 | 64.1 | 64.5 | 69.4 | 58.8 | 64.3 | 64.3 | 54.8 | 48.7 | 69.7 | 58.6 |
| SampEn | 53.5 | 75.0 | 91.2 | 25.0 | 57.1 | 74.4 | 74.2 | 78.4 | 69.7 | 74.3 | 89.3 | 69.1 | 65.8 | 90.6 | 77.1 |
| CTM | 39.7 | 75.0 | 88.5 | 20.5 | 45.7 | 28.2 | 64.5 | 50.0 | 41.7 | 44.3 | 32.1 | 64.3 | 37.5 | 58.7 | 51.4 |
| LZC | 46.6 | 66.7 | 87.1 | 20.5 | 50.0 | 66.7 | 71.0 | 74.3 | 62.9 | 68.6 | 92.9 | 66.7 | 65.0 | 93.3 | 77.1 |
| ODI3 | 64.7 | 83.3 | 95.7 | 29.4 | 77.1 | 74.4 | 74.2 | 78.4 | 69.7 | 74.3 | 89.3 | 69.1 | 65.8 | 90.6 | 77.1 |

Se: sensitivity (%);
Sp: specificity (%);
PPV: positive predictive value (%);
NPV: negative predictive value (%);
Acc: accuracy (%) Features with the highest accuracy for each OSAHS cut-off are highlighted in bold

TABLE IV
DIAGNOSTIC PERFORMANCE IN THE TEST SET OF EACH OPTIMUM OXIMETRIC
MODEL FROM FCBF USING DIFFERENT CUT-OFF POINTS

| | | cut-off AHI = 1 e/h | | | | | cut-off AHI = 3 e/h | | | | | cut-off AHI = 5 e/h | | | | |
|---|---|---|---|---|---|---|---|---|---|---|---|---|---|---|---|---|
| Performance (%) | | Se | Sp | PPV | NPV | Acc | Se | Sp | PPV | NPV | Acc | Se | Sp | PPV | NPV | Acc |
| Log | LDA | 53.5 | 91.7 | 96.9 | 29.0 | 60.0 | 53.9 | 93.6 | 91.3 | 61.7 | 71.4 | 67.9 | 100.0 | 100.0 | 82.4 | 87.1 |
| criterion | QDA | 46.6 | 91.7 | 96.4 | 26.2 | 54.3 | 38.5 | 90.3 | 83.3 | 53.8 | 61.4 | 71.4 | 100.0 | 100.0 | 84.0 | 88.6 |
| | LR | 91.4 | 8.3 | 82.8 | 16.7 | 77.1 | 61.5 | 87.1 | 85.7 | 64.3 | 72.9 | 85.7 | 88.1 | 82.8 | 90.2 | 87.1 |

Se: sensitivity (%);
Sp: specificity (%);
PPV: positive predictive value (%);
NPV: negative predictive value (%);
Acc: accuracy (%) Features with the highest accuracy for each OSAHS cut-off are highlighted in bold

### A.4 Discussion and Conclusions

Feature extraction, selection, and classification algorithms were assessed in the context of screening for pediatric OSAHS using SpO2 recordings obtained during overnight polysomnographic evaluations in a clinical setting. All feature extraction approaches (time, frequency, linear, and nonlinear) were present in all optimum feature subsets from FCBF, suggesting the complementarity of the proposed methods. Our results suggest that M2t, PA, SampEn, and ODI3 are relevant for the disease because they were always selected. Similarly, M2t, PA, and ODI3 achieved the highest individual performance using the cut-off points 5, 3, and 1 e/h, respectively. Optimum pattern recognition models improved individual features for a cut-off AHI=5 e/h. The highest performance was reached by QDA, which achieved 71.4% Se, 100.0% Sp and 88.6% Acc in the test set. It is important to point out that using this model there are no false negatives: if children test positive, then they definitely have OSAHS (positive post-test probability of 100%).

Our results agree with recent studies focused on screening methods for OSAHS in children. The study by Sahadan et al. analyzed a population of 93 children and achieved 18% Se and 97% Sp (cut-off AHI=1 e/h) using pulse rate conventional measures from pulse oximetry recordings [12]. Similarly, the study by Garde et al. used SpO2 and pulse rate from a population composed of 146 children. The proposed LDA model achieved 88.4% Se and 83.6% Sp (cut-off AHI=5 e/h) in a test set [6]. In [1], Kadmon et al. assessed a simplified sleep-related questionnaire for screening OSAHS in a population of 85 children. Their method achieved 83% Se and 64% Sp (cut-off AHI=5 e/h).

The population cohort evaluated herein can be expanded in order to derive more generalizable conclusions. In addition, input parameters of spectral and nonlinear analyses can be thoroughly optimized. Finally, additional feature selection and classification methods can potentially be assessed.

In summary, our results suggest that the methodology of the present invention of automated analysis of overnight SpO2 using suitable features and statistical pattern recognition models can improve the performance of oximetry as a screening tool for OSAHS in children.

B. Analysis and Classification of Oximetry Recordings to Predict Obstructive Sleep Apnea Severity in Children

According to the next aspect of the present invention, the use of the information contained in a single-channel SpO2 is employed for OSAHS severity detection. The utilization of data from the SpO2 channel simplifies the OSAHS diagnosis and severity assessment in children. Hence, the main objective of this aspect of the method of the present invention is to evaluate the diagnostic ability of the information contained in the SpO2 signal. Specifically, the spectrum of SpO2 recordings from children is analyzed and divided into three groups according to their corresponding AHI. There exists a lack of consistency in the literature as to the optimal AHI cutoff to determine OSAHS in children, with most of the studies applying 1, 3, or 5 events per hour (e/h) [4]. Here AHI<1 e/h was employed as the most restrictive cutoff to discard OSAHS and AHI≥5 e/h to define a group with the highest OSAHS severity. Additionally, another group was formed with those patients in the range 1≤AHI<5 e/h, which is recognized as the most challenging concerning the decision to implement treatment, usually consisting of surgical removal of tonsils and adenoids. Therefore, evaluation was done of the spectrum of the SpO2 recordings from children in the three groups looking for discriminative features. Additionally, 3% ODI (ODI3) for comparison purposes was utilized. Finally, the spectral information and ODI3 was combined by means of an artificial neural network, a multi-layer perceptron (MLP), in order to classify children into one of the three groups. This multiclass approach allows the definition of a protocol which includes doubtful subjects, as well as allows the evaluation, at the same time, of both AHI=1 e/h and AHI=5 e/h cutoffs from a binary classification point of view.

### B.2 Subjects and Signals Under Study

As before, the study involved SpO2 recordings from 176 children (97 males and 79 females). All of them were clinically referred to the Pediatric Sleep Unit at the University of Chicago Medicine corner Children's Hospital (Chicago, Ill., USA) due to suspicion of OSAHS. The Ethical Committee approved the protocol and an informed consent to participate in the study was obtained for each child. Overnight PSGs were conducted from 20:00 to 08:00. Recordings were acquired by means of a digital polysomnography system (Polysmith; Nihon Kohden America Inc., CA, USA). Detection and quantification of sleep and cardiorespiratory events were carried out according to the rules of the American Academy of Sleep Medicine. Thus, apnea was defined as the absence of oronasal airflow during at least 2 respiratory cycles. Accordingly, hypopnea was defined as a decrease ≥30% in the nasal pressure airflow signal lasting at least 2 respiratory cycles, leading to a desaturation ≥3% and/or an arousal. As previously stated, children were divided into three groups according to their corresponding AHI: AHI under 1 e/h (ABI_{□}1), AHI in the range [1, 5) e/h (AHI[1,5)), and AHI equal or above 5 e/h (AHI≥5). Table V summarizes demographic and clinical data from subjects according to this division. No statistical significant differences (p-value<0.01) were found in age, gender, and body mass index (BMI) when applying the non-parametric Kruskal-Wallis test to compare the three groups.

The SpO2 recordings were acquired during PSG at a sampling rate of fs=25 Hz. Artifacts due to children movements were automatically removed during preprocessing. Thus, SpO2 values equal to zero as well as differences between consecutive SpO2 samples ≥4% were considered artifacts. Removed samples were substituted by interpolated data. ODI3 was estimated as the number of desaturations (at least 3%) per hour of sleep time.

TABLE V
DEMOGRAPHIC AND CLINICAL

| | All | AHI_{<1} | AHI_{[1,5)} | AHI_{≥5} |
|---|---|---|---|---|
| # Subjects | 176 | 30 | 75 | 71 |
| Age⁺ (years) | 7.0 ± 3.6 | 8.2 ± 3.3 | 7.3 ± 3.5 | 6.1 ± 3.6 |
| Male (%) | 55.1 | 56.7 | 54.7 | 54.9 |
| BMI* (kg/m²) | 20.6 ± 7.3 | 20.5 ± 6.8 | 20.6 ± 6.7 | 20.7 ± 8.2 |
| AHI (e/h) | — | 0.5 ± 0.3 | 2.6 ± 1.1 | 19.3 ± 23.1 |

BMI: Body Mass Index;
AHI: Apnea Hypopnea Index;
⁺p-value = 0.016;
*p-value = 0.816

### B.3 Methodology

The methodology was divided into three steps. First, a spectral analysis of the SpO2 recordings was conducted to look for differences among the three groups. Then several spectral features were extracted according to this analysis. Finally, the spectral data and ODI3 were combined through MLP to classify the children into one of the three classes.

### B.3.1 Spectral Analysis and Feature Extraction

Power spectral density (PSD) was estimated for each SpO2 recording by means of the Welch's method [15]. A Hamming window of 2¹³ samples (5.5 minutes), 50% overlap, and a discrete Fourier transform of 2¹⁴ samples was used. FIG. 1 shows the median PSD for each group of OSAHS severity. Higher PSDs can be observed as the severity increases. A band of interest (BW) is also shown in the range 0.01370.0473 Hz. This corresponds to the spectral bandwidth in which the three groups showed statistical significant differences (Mann-Whitney U test) in their PSD amplitude values (p-value=0.01, p-value=0.0033 after Bonferroni correction). In this case, BW is equivalent to the bandwidth in which AHI<1 and AHI[1,5) showed significant differences. FIG. 2 displays the p-value vs. frequency plots for each of the three possible comparisons. The limits of BW are easily located as the crosspoints between the AHI<1 vs. AHI[1,5) p-value (f) curve and the p-value significance level line.

The following features were extracted from the BW of each PSD: maximum PSD value (MA), minimum PSD value (mA), spectral power (PS, as the area under the PSD at BW), and standard deviation of the PSD values (SDf). According to FIG. 1, higher values were expected in these features as the OSAHS severity increases. After feature extraction, each subject under study is characterized by a vector xi (i=1, 2 . . . M, M=176) whose 5 components are the corresponding values of the four spectral features and ODI3.

### B.3.2 Multi-Layer Perception

MLP is a supervised learning algorithm whose architecture is arranged in several interconnected layers (input, hidden, and output). These are composed of units known as neurons or perceptrons. Each neuron is characterized by an activation function g(•) and their connections to neurons from other layers (wi, j). Here, the input layer had five units, corresponding to the number of spectral features obtained for each subject (MA, mA, PS, SDf) and ODI3. Moreover, since the purpose is to carry out a three-class classification, three output units with a logistic activation function were used. A single hidden layer was implemented, composed of neurons with non-linear activation functions. This configuration is known to be able to provide a universal function approximation. Since the number of neurons in the hidden layer (NH) controls the effective complexity of the network, a small number, NH=5, was chosen to prevent network from overfitting. Thus, the final input-layer:hidden-layer:output-layer architecture was 5:5:3 neurons. The weights wi, j were optimized using the sum of squares error function minimization criterion by means of the scaled conjugate gradient algorithm. For each subject under study, the final classification task was performed by assigning the corresponding xi (i=1, 2 . . . M, M=176) to the class with the highest probability in the output layer.

### B.3.3 Statistical Analysis

The non-parametric Kruskal-Wallis test was used to assess statistical differences in the spectral features from the OSAHS severity groups. A confusion matrix was used to evaluate the performance of multiclass MLP. Also, to assess the output of MLP from a binary classification point of view, sensitivity (Se, percentage of OSAHS-positive subjects rightly classified), specificity (Sp, percentage of OSAHS-negative subjects rightly classified), accuracy (Acc, overall percentage of subjects rightly classified), positive predictive value (PPV, proportion of positive test results which are true positives), negative predictive value (NPV, proportion of negative test results which are true negatives), positive likelihood ration (LR+, Se/(1−Sp)), and negative likelihood ratio (LR−, (1−Se)/Sp) measured the diagnostic ability for both AHI=1 e/h and AHI=5 e/h cutoffs. All these statistics were obtained after leave-one-out cross-validation (loo-cv).

### B.4 Results

Table VI displays the values of the spectral features and ODI3 for each of the three OSAHS severity groups (mean±standard deviation). All of them showed large statistical significant differences when comparing the three groups by means of Kruskal-Wallis test. As expected, the five features are higher as the OSAHS severity increases.

Table VII shows the confusion matrix resulting from the diagnostic ability assessment of the MLP network for the three-class classification task (results after loo-cv). A total of 125 out of 176 subjects were rightly classified in their actual class (71.0%). Per classes, 50.0% (15 out of 30) of the subjects in AHI<1, 80.0% (60 out of 75) in AHI[1,5), and 70.4% (50 out of 71) in AHI≥5 were rightly classified.

Table VIII shows the diagnostic ability of MLP and ODI3 when assessing both the AHI=1 e/h and AHI=5 e/h cutoffs (results after loo-cv). MLP results are directly derived from the confusion matrix. For both cutoffs the global Acc of MLP is higher than the corresponding ODI3 (84.7% vs. 78.4% and 85.8% vs. 76.7%, respectively). In the case of AHI=1 e/h, ODI3 is much more specific than MLP, leading to higher PPV and LR+. In the case of AHI=5 e/h, however, MLP outperforms ODI3 at each statistic.

TABLE VI
VALUES OF THE SPECTRAL FEATURES AND ODI₃
(MEAN ± STANDARD DEVIATION)

| Features | AHI_{<1} | AHI_{[1,5)} | AHI_{≥5} | p-value |
|---|---|---|---|---|
| ODI₃ (e/h) | 1.01 ± 1.10 | 3.21 ± 2.80 | 16.18 ± 20.78 | <10⁻¹⁷ |
| MA (W/Hz) | 7.76 ± 7.41 | 13.58 ± 16.98 | 71.61 ± 149.83 | <10⁻¹² |
| mA (W/Hz) | 1.44 ± 0.59 | 2.91 ± 5.23 | 19.51 ± 70.43 | <10⁻¹¹ |
| Ps (W) (10⁻¹) | 7.80 ± 4.32 | 15.15 ± 21.02 | 97.74 ± 248.19 | <10⁻¹³ |
| SD_{f} (W/Hz) | 1.84 ± 1.81 | 3.23 ± 4.12 | 16.31 ± 27.29 | <10⁻¹¹ |

### B.5 Discussion and Conclusions

In this aspect of the invention, an automatic diagnostic methodology for pediatric OSAHS severity based on the information contained in single-channel SpO2 was developed. Features from a spectral band of interest and the clinical variable ODI3 were combined by means of MLP to classify subjects into one out of the three OSAHS severity levels.

The spectral analysis of the SpO2 signal revealed a band of interest (BW=0.0137-0.0473 Hz) in which statistically significant differences were found for the three classes. The lower limit of BW is consistent with the corresponding band of interest in adults (0.014-0.033 Hz, i.e., events lasting from 30 to 71 s). Conversely, a higher upper limit was found in children, suggesting shorter events as also significant for them. This agrees with the higher respiratory rate reported in children. However, further analysis is required regarding the causes of the differences in both bands.

The spectral features extracted from BW showed statistically significant differences when comparing the three classes. All of them reached higher values as the OSAHS severity increases. Since the ideal SpO2 time series is a constant, close to 100%, the higher PSD values in the frequencies correspond to more desaturations and recoveries to the baseline. Consequently, higher MA, mA, PS, and SDf suggest more desaturation events both in discrete frequencies (MA, mA) and in the whole band (PS, SDf), which is consistent with the clinically used severity classification of OSAHS.

The multiclass MLP proposal correctly classified 71% of the subjects. Although this overall accuracy is arguably not quite high enough, a deeper study of the subjects wrongly classified reveals that the 11 children who belong to AHI[1,5), and were assigned to AHI<1, present an AHI of 1.65±0.42 e/h. This means that 96.3% of subjects predicted as AHI<1 have no OSAHS or a low severity degree. Additionally, the 4 children from AHI[1,5) assigned to AHI≥5 present an AHI of 3.0±1.7 e/h, i.e., 100% of children predicted as AHI≥5 have severe OSAHS or a higher severity degree comparing with the mean of the AHI[1,5) class. Finally, children assigned to AHI[1,5) come from the three classes: AHI<1 (15.8%), AHI[1,5) (63.2%), and AHI≥5 (21.0%). Consequently, subjects assigned to this class should be regarded as inconclusive. A screening protocol could be generated from these results as follows: i) if MLP predicts AHI<1, discard OSAHS; ii) if MLP predicts AHI≥5, consider treatment; iii) if MLP predicts AHI[1,5), send to overnight PSG. Since the SpO2 signal is easily acquired from an oximeter, such a protocol would reduce the need by 46% (81/176) of overnight PSGs.

Other studies analyzed physiological signals to help in pediatric OSAHS diagnosis. All of them reported results from a binary classification point of view. One study analyzed 50 ECG recordings, reaching 85.7% Se, 81.8% Sp, and 84.0% Acc using a quadratic linear discriminant applied to 23 features (AHI cutoff=1 e/h). Another assessed the diagnostic ability of information contained in 21 PPG time series, reporting 75.0% Se, 85.7% Sp, and 80.0% Acc (AHI cutoff=5 e/h). Yet another combined spectral features from 50 AF recordings with ODI3 from SpO2 to achieve 85.9% Se, 87.4% Sp, and 86.3% Acc with a logistic regression methodology (AHI cutoff=3 e/h). Finally another reported 83.6% Se, 88.4% Sp, and 85.0% Acc in a 146 subject database by combining 8 features from SpO2 and PRV in a linear discriminant [11]. The present MLP methodology can be assessed for AHI=1 e/h and AHI=5 e/h at the same time (84.7% and 85.8% Acc, respectively).

Although the number of subjects is not small when comparing to other similar studies, more children, particularly those with AHI<1 e/h, would be necessary for the sake of a more robust MLP training. This could include a training-test strategy as well as the evaluation of a range of neurons in the hidden layer, which was arbitrary set to a low value in order to decrease the chances for overfitting.

Additionally, more subjects would also let us use a training set from which could be independently obtained the spectral band of interest. However, a loo-cv methodology was used to validate our results. Finally, the use of features from time domain could complement the findings of the present invention. The assessment of features and classification models other than those presented in this work using a larger dataset is contemplated.

In summary, a multiclass MLP methodology was developed with capability to help in pediatric OSAHS severity screening. The SpO2 features obtained from a frequency band of interest, combined with ODI3 through MLP, outperform the single diagnostic yield of this clinical variable. This can be also evaluated for binary classification purposes, reaching high diagnostic ability comparing with recent state-of-the-art studies. Thus, the results suggest that the information contained in single-channel SpO2 is helpful to detect severity categories among children with OSAHS that are worthy of mention.

We claim:

1. A system and method of diagnosing obstructive sleep apnea as described herein, in any embodiment and any configuration.

2. A system and method to detect and measure the presence and severity of pediatric sleep apnea using at least one of a computer analytic system, neural networks, and artificial intelligence and further comprising a pulse oximeter for measuring patient blood oxygen saturation.
